(19) United States
(12) Patent Application Publication
(10) Pub. No.: US 2009/0056793 A1
(43) Pub. Date: Mar. 5, 2009

(54) BENZOTERRYLENE DERIVATIVES

(75) Inventors: Heinz Langhals, Ottobrunn (DE); Simon Poxleitner, Munchen (DE)

Correspondence Address: SABIC - LEXAN;SABIC Innovative Plastics - IP Legal, ONE PLASTICS AVE., PITTSFIELD, MA, 01201-3697, US

(73) Assignee: SABIC Innovative Plastics IP BV, Bergen op Zoom (NL)

(21) Appl. No.: 11/848,490

(22) Filed: Aug. 31, 2007

(51) Int. Cl. H01L 31/04; C07D 471/02; C07D 471/12; C07D 471/22; C07D 491/12; C07D 491/22; C08K 5/1539; C08K 5/3415

(52) U.S. Cl. 136/252; 524/112; 524/90; 546/26; 546/27

(57) ABSTRACT

A benzoterrylene of Formula (I):

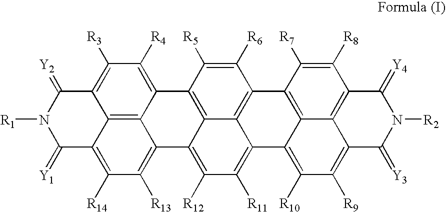

wherein at least one of the pairs R₄-R₅, R₆-R₇, R₁₀-R₁₁, and R₁₂-R₁₃ is a ring structure selected from the group consisting of Formulas A, B, and C:

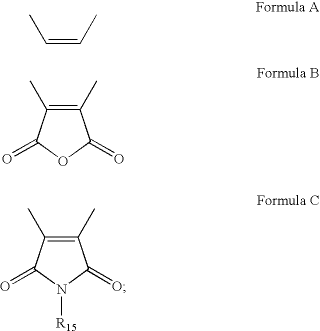

wherein Y₁ through Y₄ are each independently selected from O and NR₁₆; and R₁ through R₁₆ are as disclosed herein. The benzoterrylenes are useful as lightfast colorants with high fluorescence quantum yields. Also disclosed are methods of making and using the benzoterrylenes.

### BACKGROUND

The present disclosure relates to benzoterrylene tetracarboxylic bisimide derivatives. Methods of making and using such derivatives, such as for use as colorants, are also disclosed, as well as compositions and articles comprising the same.

Perylene carboxylic bisimides are useful as lightfast colorants. They are suitable as pigments and fluorescent dyes with absorption in the cyan-green region of the electromagnetic spectrum and fluorescence in the long-wavelength red region.

In the case of perylene tetracarboxylic acid bisimides, it is possible to obtain soluble lightfast fluorescent colorants that fluoresce with a quantum yield of about 100%. In this regard, certain chemical groups can be placed on the nitrogen atoms. For example, 5-di-tert-butylphenyl-, 2,5-di-isopropylphenyl- or long chain sec-alkyl groups, so-called swallowtail substituents, like 1-hexylheptyl- or 1-nonyldecyl-groups, achieve such quantum yields in solution.

It would seem that adding the same chemical groups to higher homologues of perylene, e.g. terrylene and quaterrylene, would obtain fluorescent colorants that absorb at longer wavelengths. However, only partial results are obtained. Although absorption and fluorescence occur at longer wavelengths, a lower fluorescence quantum yield is obtained. According to F. Nolde, Jianquiang Qu, C. Kohl, N. G. Pschirer, E. Reuther and K. Muellen, Chem. Eur. J., 2005, 11, 3959-3967, the fluorescence quantum yield for terrylene carboxylic bisimides, for example, dropped to about 60%.

For luminescent solar collectors (LSC) and other applications, there is a need for fluorescent colorants that absorb at longer wavelengths but retain a high quantum yield.

### BRIEF DESCRIPTION

Disclosed, in various embodiments, are benzoterrylene derivatives and processes for making and using them. They absorb light at longer wavelengths and have a higher quantum yield. In some embodiments, the benzoterrylene derivatives can be used as colorants for coloring organic and inorganic materials. They also can be used in the production of colored compositions and/or the fabrication of devices comprising the same.

In embodiments, a benzoterrylene has the structure of Formula (I):

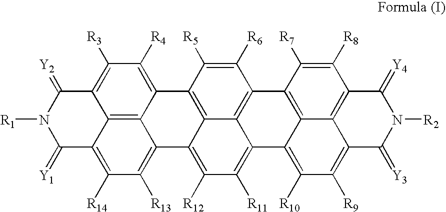

wherein at least one of the pairs R₄-R₅, R₆-R₇, R₁₀-R₁₁, and R₁₂-R₁₃ is a ring structure selected from the group consisting of Formulas A, B, and C:

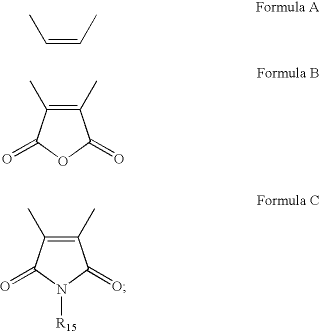

wherein Y₁ through Y₄ are each independently selected from O and NR₁₆, and R₁ or R₂ may independently combine with R₁₆ to form a ring structure selected from the group consisting of Formulas D and E:

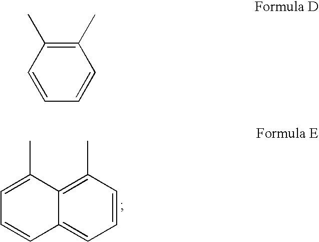

wherein R₁, R₂, R₁₅, and R₁₆ are each independently selected from hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono; and

wherein R₃ through R₁₄ are each independently selected from halogen, cyano, hydroxyl, hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono.

In some embodiments, the ring structure is formed at the R₄-R₅ pair and is of Formula C.

In other embodiments, two ring structures are formed at the R₄-R₅ pair and the R₁₀-R₁₁ pair, and both ring structures are of Formula C.

In some embodiments, the ring structure is formed at the R₄-R₅ pair and is of Formula B.

In other embodiments, two ring structures are formed at the R₄-R₅ pair and the R₁₀-R₁₁ pair, and both ring structures are of Formula B.

In some embodiments, R₁, R₂, and R₁₅ are independently selected from secondary alkyl having from about 10 to about 25 carbon atoms and R₃ through R₁₄ are hydrogen.

The benzoterrylene may have a fluorescent quantum yield of at least 70%. In further embodiments, the fluorescent quantum yield is at least 80% or at least 90%.

A composition may be formed, comprising a polymeric resin and the benzoterrylene.

A dye or pigment may comprise the benzoterrylene.

A method of coloring a polymeric resin may comprise the step of incorporating the benzoterrylene into the polymeric resin.

An article may be molded from a composition, the composition incorporating the benzoterrylene.

A luminescent solar collector may comprise: a sheet which comprises a polymer and the benzoterrylene of claim 1; and a light energy converter which is operatively connected to the sheet.

A method of preparing a benzoterrylene of Formula (I) is also disclosed, comprising:

reacting a naphthalene-1,8-dicarboximide of the general formula

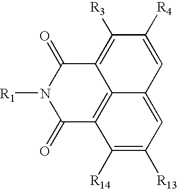

with a perylene-3,4-dicarboximide of the general formula

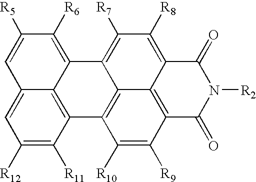

to form a terrylene tetracarboxylic bisimide; and

reacting the terrylene tetracarboxylic bisimide with a dienophile to obtain the benzoterrylene.

The dienophile may be an unsaturated dicarboxylic acid or a dicarboxylic acid anhydride, such as maleic acid or maleic anhydride.

The method may further comprise reacting the benzoterrylene with an amine to obtain a benzoterrylene hexacarboxylic trisimide. A dibenzoterrylene octacarboxylic tetraimide may also be obtained.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates a first step in a process for making the benzoterrylene derivatives of the present disclosure.

FIG. 2 illustrates a second step in a process for making the benzoterrylene derivatives of the present disclosure.

FIG. 3 illustrates a third step in a process for making the benzoterrylene derivatives of the present disclosure.

FIG. 4 illustrates a fourth step in a process for making the benzoterrylene derivatives of the present disclosure.

FIG. 5 is a graph showing the absorption spectra for a comparative compound of Structure 1 and compounds 5, 8, and 9.

FIG. 6 is a graph showing the absorption spectra for a comparative compound of Structure 1 and the absorption and emission spectra for compound 8.

FIG. 7 is a graph showing the absorption spectra for a comparative compound of Structure 1 and the absorption and emission spectra for compound 9.

FIG. 8 is a graph showing the absorption spectra for a comparative compound of Structure 1 and the absorption and emission spectra for compound 5.

### DETAILED DESCRIPTION

A more complete understanding of the compositions and processes disclosed herein can be obtained by reference to the accompanying drawings. These drawings are merely schematic representations based on convenience and the ease of demonstrating the present disclosure, and are, therefore, not intended to indicate relative size and dimensions of the devices or components thereof and/or to define or limit the scope of the exemplary embodiments.

Perylene tetracarboxylic bisimides have the following general structure 1:

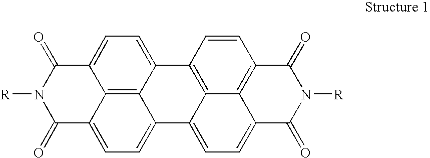

wherein R is as previously described.

In contrast, the benzoterrylenes of the present disclosure have the following Formula (I):

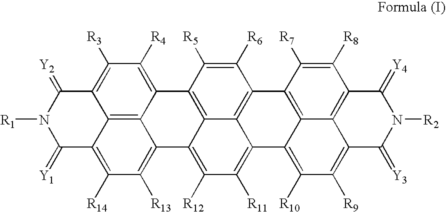

wherein at least one of the pairs R₄-R₅, R₆-R₇, R₁₀-R₁₁, and R₁₂-R₁₃ is a ring structure selected from the group consisting of Formulas A, B, and C:

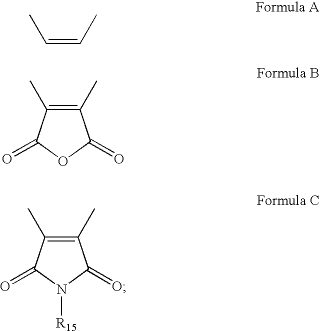

wherein Y₁ through Y₄ are each independently selected from O and NR₁₆, and R₁ or R₂ may independently combine with R₁₆ to form a ring structure selected from the group consisting of Formulas D and E:

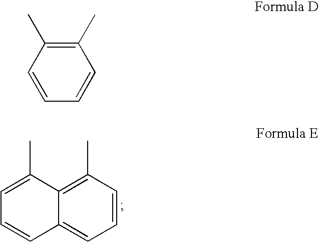

wherein R₁, R₂, R₁₅, and R₁₆ are each independently selected from hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono; and

wherein R₃ through R₁₄ are each independently selected from halogen, cyano, hydroxyl, hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono.

The term “alkyl” should be construed as including at least linear and branched variants. In specific embodiments, R₁ and R₂ are independently selected from secondary alkyl having from about 10 to about 25 carbon atoms. In other specific embodiments, R₁ and R₂ are independently selected from 2,6- and 2,5-dialkylphenyl groups wherein the alkyl groups independently comprise up to 8 carbon atoms. Again, the alkyl groups may be linear or branched.

In some embodiments, the benzoterrylene has one of the following Formulas (II), (III), (IV), or (V):

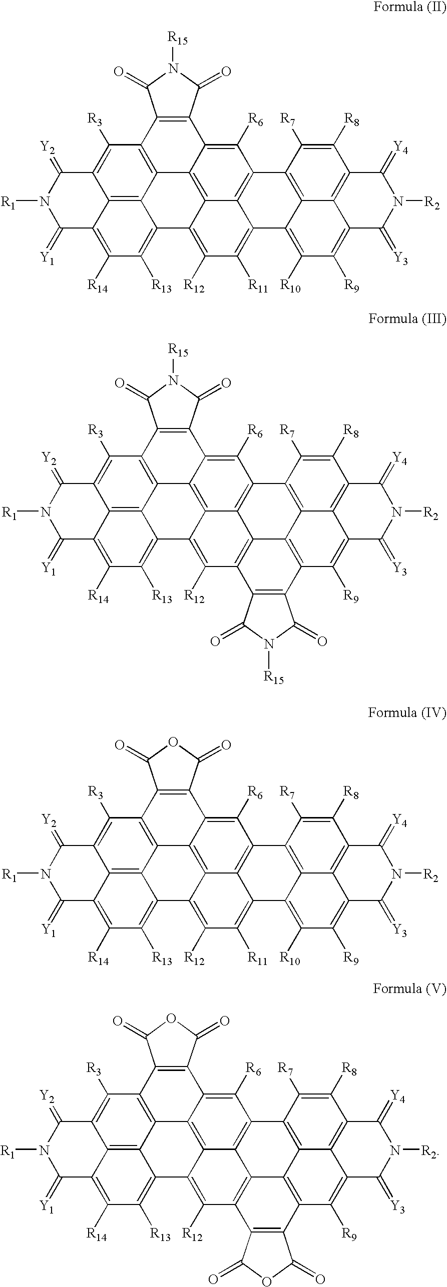

In other embodiments, the benzoterrylene is one of Formulas (II), (III), (IV), or (V), wherein R₃ through R₁₄ are hydrogen, and R₁, R₂, and R₁₅ are independently selected from secondary alkyl having from about 10 to about 25 carbon atoms. These embodiments are reflected in the benzoterrylenes of Formulas (VI), (VII), (VIII), and (IX):

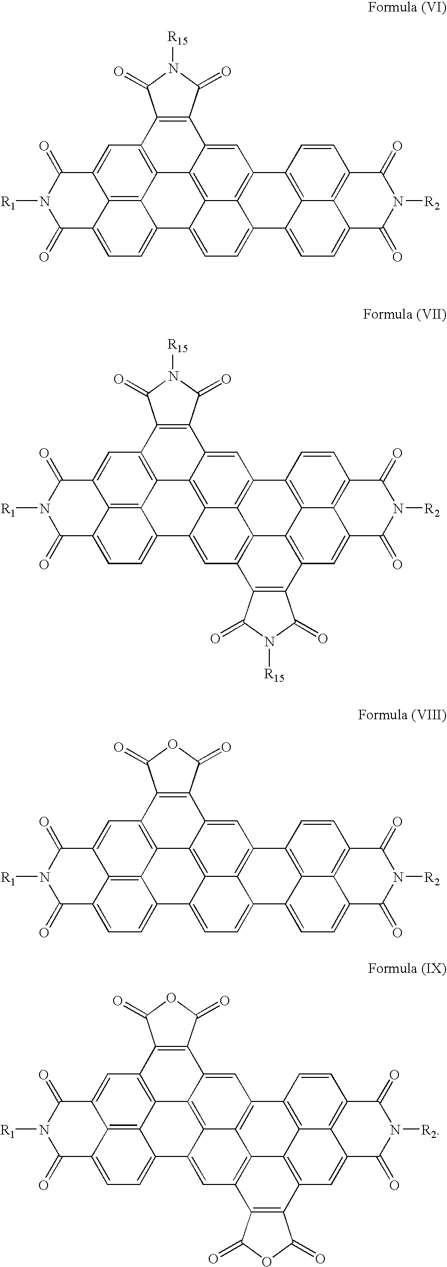

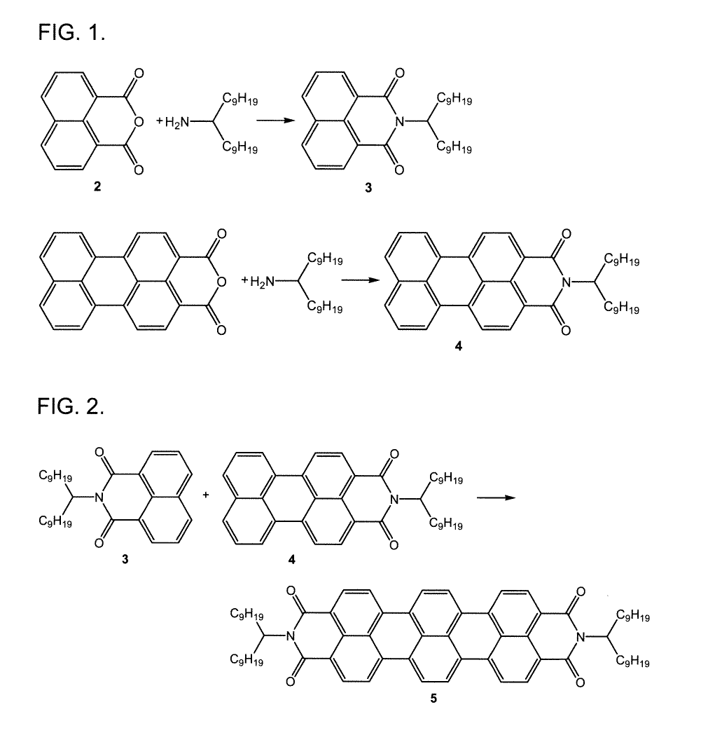

The benzoterrylenes of the present disclosure can be made by various methods. One method is illustrated in FIGS. 1-4. The synthesis of benzoperylene carboxylic bisimides from perylene carboxylic bisimides via Clar-type Diels-Alder reaction is known. This reaction requires, however, comparatively severe reaction conditions of heating at 175° C. for five days.

As shown in FIG. 1, suitable starting materials can be obtained by reacting a long chain amine, such as 1-nonyidecylamine, with naphthalene-1,8-dicarboxylic acid anhydride 2 to form the corresponding naphthalene-1,8-dicarboximide 3. The same reaction of a long chain amine with a perylene-3,4-dicarboxylic acid anhydride forms a perylene-3,4-dicarboximide 4. This reaction can be performed under atmospheric pressure at temperatures of 110° C. to 160° C. for generally at least two hours.

The naphthalene-1,8-dicarboximide 3 and perylene-3,4-dicarboximide 4 can then be reacted together under mild conditions, as described by T. Sakamoto, C. Pac, J. Org. Chem., 66:94-98 (2001), to form a terrylene tetracarboxylic bisimide 5. This reaction is shown in FIG. 2. This reaction can be performed under atmospheric pressure at temperatures of 130° C. to 160° C. for usually at least three hours. The terrylene tetracarboxylic bisimide 5 has surprisingly good solubility so that it is suitable for a Diels-Alder reaction.

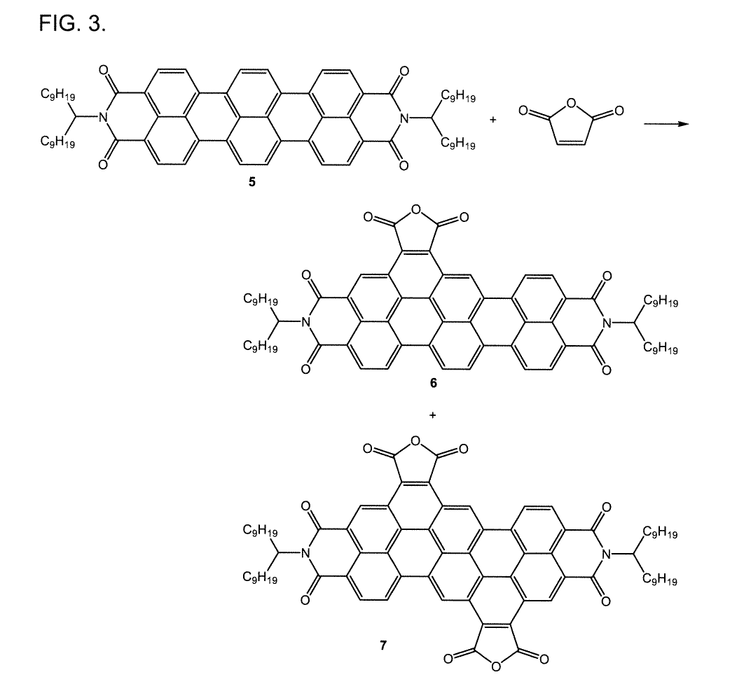

As shown in FIG. 3, the terrylene tetracarboxylic bisimide 5 can then be reacted with an unsaturated dicarboxylic acid or its anhydride, such as maleic anhydride, in a nitrobenzene solution to form a benzoterrylene hexacarboxylic bisimide mono adduct 6 and/or dibenzoterrylene octacarboxylic bisimide double adduct 7. Quite surprisingly, this reaction takes place at 210° C. within hours, not days as was expected, and is evident by a change in color of the solution. The reaction can be performed under atmospheric pressure at temperatures above 200° C. for a couple of hours, usually more than two hours.

Nitrobenzene can act as a mild oxidizing agent, in the process becoming reduced to aniline. The aniline can react with the anhydride groups to form N-phenyl carboxylic imides. This may be a problem if other carboxylic imide groups are desired. However, the formation of N-phenyl carboxylic imides can be prevented by the addition of chloranil (a stronger oxidizing agent) and by adding the maleic anhydride in far more than stoichiometric quantities. In embodiments, the maleic anhydride is added in a ratio of about 10 to about 50 times the molar quantity of the terrylene tetracarboxylic bisimide. If, however, N-phenyl carboxylic imides are desired, it is better to prepare the adducts first and then convert the intermediates completely with aniline.

It is generally difficult to remove the nitrobenzene solvent completely from the reaction mixture by ordinary distillation or by chromatographic methods. However, it was found that the nitrobenzene could be removed easily and completely by means of a water vapor distillation.

After distillation, a product mixture comprises, as main products, the mono adduct 6 and the double adduct 7. These two components can be separated with some effort to obtain benzoterrylene carboxylic bisimides substituted with Formula C as described by Formula (I) above.

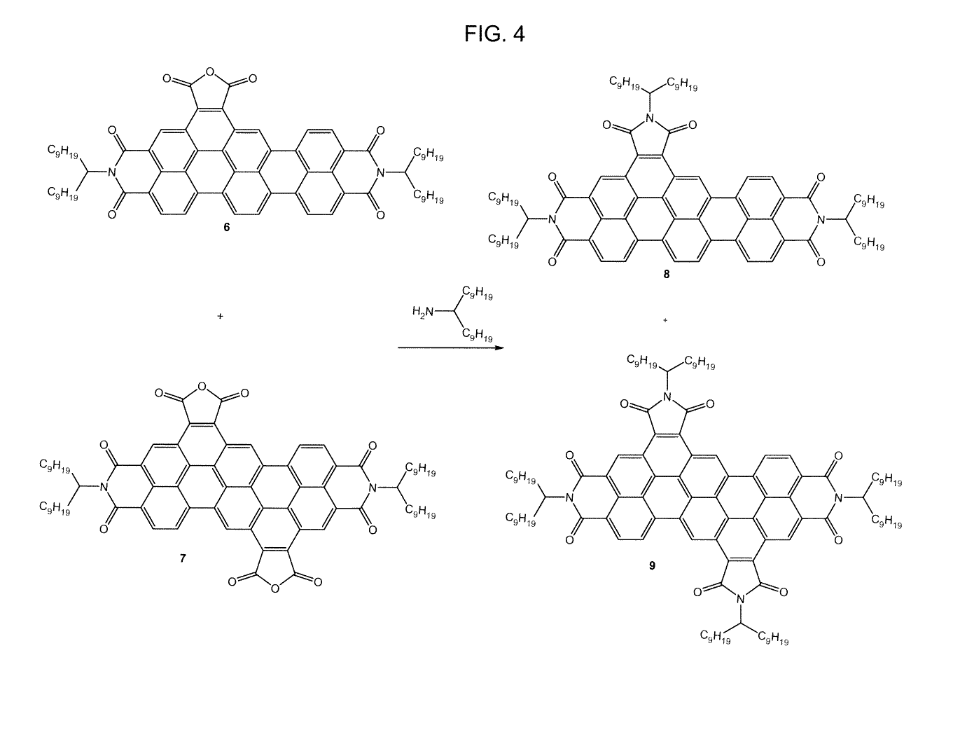

Alternatively, the mono adduct 6 and double adduct 7 can be further reacted with a long chain amine, such as 1-nonyldecylamine, to obtain the benzo trisimide adduct 8 and dibenzo tetraimide adduct 9. The reaction can be performed under atmospheric pressure at temperatures above 200° C. within a couple of hours, usually more than two hours. This is shown in FIG. 4. These two adducts are very soluble and can be isolated by chromatographic means. As described above, a benzoterrylene corresponding to Formula (I) with either Formula B or C can be obtained. To obtain a benzoterrylene with Formula A, decarboxylation of 6 and 7 is required.

The benzoterrylenes of the present disclosure have good absorption at longer wavelengths. The ultraviolet/visible spectroscopy (“UV/VIS”) absorption of the benzo trisimide adduct 8 has a bathochromic shift compared to the corresponding perylene carboxylic bisimide of Structure 1 where R is a 1-hexylheptyl group and a hypsochromic shift compared to the terrylene tetracarboxylic bisimide 5.

An analogous hypsochromic shift in absorption occurs by the benzannulation of perylene carboxylic bisimides to benzoperylene carboxylic trisimides. The UV/VIS spectrum of the benzo trisimide adduct 8 also absorbs at about 400 nm; this is probably caused by the five-membered-ring carboxylic imide structure. This makes it possible to collect light over a broader range of the spectrum, thus increasing the efficiency of a light collecting device which uses the benzoterrylenes of the present disclosure. The benzoterrylenes can thus be useful as colorants in devices such as luminescent solar collectors.

The benzo trisimide adduct 8 also has strong fluorescence with a quantum yield of almost 100%. This is very surprising because Nolde had found a dramatic decrease in the quantum yields of terrylenes and quaterrylenes. This quantum yield is also a considerable increase compared to other terrylene carboxylic bisimides.

The second benzannulation in the dibenzo tetramide adduct 9 appears to cause a further hypsochromic color shift, causing absorption at shorter wavelengths, as occurs with the perylene tetracarboxylic bisimides with Structure 1. However, compared to Structure 1 (where R=1-hexylheptyl), the dibenzo tetramide adduct 9 also has considerable absorption in the short wavelength visible light and UVA range, making it possible to absorb light over a broad spectral range. The fluorescence spectrum of the dibenzo tetraimide adduct 9 is almost identical to the fluorescence spectrum of the compounds of structure 1, meaning that the Stokes shift (the difference between the wavelength of maximum absorption and the wavelength of maximum emission) has been increased in comparison with the compounds of Structure 1.

The fluorescence quantum yield of the dibenzo tetramide adduct 9 is close to 100%. In embodiments, the quantum yield is at least 70%. In further embodiments, the quantum yield is at least 80% or at least 90%.

The benzoterrylene colorants of the present disclosure also have good lighffastness as is generally known for perylene and terrylenes. This makes them suitable for many applications, such as in luminescent solar collectors.

The benzoterrylene colorants of the present disclosure can be used in several different applications. They can be used to color polymeric compositions; as dyes or pigments; in making paints, inks, coatings and the like; for security-marking purposes; for labeling objects; for converting light frequencies; for passive display elements; as starting materials for superconducting organic materials; as fluorescent dyes for machine-readable markings; as laser dyes; and for preparing non-impact printing toners, color filters, organic photoreceptors, electroluminescence and photoluminescence elements.

For example, they can be used as pigments for the mass coloration of plastics or coatings and paints. Accordingly, the present disclosure also relates to mass-coloured high-molecular-weight organic material containing a benzoterrylene of Formula (I) and a process for mass-colouring high-molecular-weight organic material using these compounds.

Examples of suitable plastics are polyolefins, polyvinyl chloride, fluoro polymers, for example polyfluoroethylene, polytrifluorochloroethylene or tetrafluoroethylene/hexafluoropropylene copolymers, silicone resins, but in particular engineering plastics, for example polycarbonates, polylacrylates, polymethacrylates, polymethylmethacrylates, polystyrene, ABS, polyesters, in particular polyalkylene terephthalates, such as polybutylene terephthalate (PBT) or polyethylene terephthalate (PET), polyamides, polyether ketones, polyurethanes, individually or in mixtures. Advantageously, the benzoterrylenes are used in a concentration of from about 0.001% to about 10%, including about 0.01% to about 5%, by weight of the polymer.

Examples of polyolefins which can be colored with the compounds according to the invention include polyethylene of high and low densities (HDPE, LDPE and LLDPE), polyisobutylene and, in particular, polypropylene, and copolymers of polyolefins with, for example, polyethers, polyether ketones, or polyurethanes. Preference is given to polypropylene.

Coloration takes place by customary methods, for example by mixing a compound according to the invention or a mixture of such compounds with the plastic granules or powder without the need of prior incorporation into a preparation and extruding the mixture to give fibres, films or granules. The latter can then be molded, for example in an injection molding process, to give articles.

The following examples are provided to illustrate the compositions and methods of the present disclosure. The examples are merely illustrative and are not intended to limit devices made in accordance with the disclosure to the materials, conditions, or process parameters set forth therein.

### EXAMPLES

### Example 1

Preparation of N-(1-nonyldecyl)-1,8-naphthalimide 3 and N-(1-nonyldecyl)-3,4-perylene dicarboxylic imide 4

The naphthalene carboxylic imide derivative 3 was prepared as seen in FIG. 1. 1-Nonyldecyl amine (673 mg, 2.38 mmol) and 1,8-naphthalene dicarboxylic anhydride (500 mg, 2.52 mmol) were heated in imidazole (2 g) for 3 hours at 130° C., subsequently cooled, and while still warm combined with a 2 M HCI/acetic acid (1:1) followed by extraction with chloroform twice. The combined organic phases were dried with (MgSO₄), the solvent was removed by vacuum, and the product was purified in a chromatography column (silicagel 60, CHCl₃/isohexane 1:1). A pale yellow, honeylike substance was obtained in a quantity of 650 mg (59%).

The pale yellow honeylike product was characterised by chromatographic analysis, ¹H NMR, ¹³C NMR and mass spectroscopy. The data was as follows:
R_{f}=0.8 (silica gel 60, CHCl₃/isohexane 1:1), ¹H NMR: (CDCl₃, 200 MHz, 23° C.): δ=8.64-8.50 (m, 2 H), 8.25-8.14 (m, 2 H), 8.80-8.69 (m, 2 H), 5.26-5.06 (m, 1 H), 2.35-2.10 (m, 2 H), 1.90-1.70 (m, 2 H), 1.40-1.02 (m, 28 H), 0.95-0.75 ppm (m, 6 H),
¹³C NMR: (CDCl₃, 150 MHz, 25.0° C.): δ=165.4, 164.3, 133.4, 131.5, 131.5, 130.8, 128.3, 126.9, 123.4, 123.7, 54.4, 32.4, 31.8, 29.5, 29.5, 29.2, 26.9, 22.6, 14.1 ppm,
MS: (GC/EI): m/s (%): 463 (10) [m³⁰ ], 336 (5) [M⁺-C₉H₁₉], 198 (100) [M⁺-C₁₉H₃₈].

N-(1-nonyldecyl)-3,4-perylene dicarboxylic imide 4 was prepared in a similar way.

Preparation of N,N-bis-(1-nonyldecyl)-3,4:11,12-terrylene tetra carboxylic bisimide 5

N-(1-nonyldecyl)-3,4-perylene dicarboxylic imide 4 (1.00 g, 1.70 mmol) was combined under argon with potassium-tert-butylate (3.64 g, 32.4 mmol), 1,5-diazabicyclo[4.3.0]non-5-ene (4.86 ml, 40.7 mmol) and diglyme (4.00 mL) and then heated to 130° C. N-(1-nonyldecyl)-1,8-naphthalimide 3 (1.50 g, 3.23 mmol) was gradually added through a syringe over 6 hours followed by three hours stirring at 130 ° C., cooling down, pouring on water (200 mL), stirring for one hour, degassing, and drying in air (100° C.). The obtained product (250 mg, 15%) was purified in a chromatography column (silicagel, chloroform/isohexane 3:1). This reaction is schematically shown in FIG. 2.

The product was characterised by chromatographic analysis, ¹H NMR, ¹³C NMR and mass spectroscopy. Its UV/VIS spectrum and its fluorescence spectrum were measured. The data was as follows:
R_{f}=0.7 (CHCl₃),
¹H NMR: (CDCl₃, 600 MHz, 25.0° C.): δ=8.61 (d, ³J=17.8 Hz, 4 H), 8.50 (s, 4 H), 8.46 (d, ³J=7.9 Hz, 4 H), 5.25-5.18 (m, 2 H), 2.32-2.24 (m, 4 H),
1.93-1.86 (m, 4 H), 1.40-1.16 (m, 56 H), 0.83 ppm (t, ³J=8.0 Hz, 12 H),
¹³C NMR: (CDCl₃, 150 MHz, 25.0° C.): δ=164.9, 163.9, 135.4, 131.8, 131.0, 130.9, 129.8, 128.6, 125.9, 124.1, 122.5, 121.8, 121.3, 104.8, 54.6, 32.4, 31.9, 29.7, 29.6, 29.6, 29.3, 27.0, 22.7, 14.1 ppm,
UV/VIS: (Chloroform): λₘₐₓ (Eᵣₑₗ): 651 (100), 598 (51), 555 nm (17),
Fluorescence: (CHCl₃): λₘₐₓ (Iᵣₑₗ): 668 (100), 730 nm (26),
MS: (DEP/EI): m/s (%): 1047 (100) [M⁺], 781 (36) [M³⁰- C₁₉H₃₈], 514 (45) [M⁺-2x C₁₉H₃₈].

Preparation of N,N′N″-Tris-(1-nonyldecyl)benzo[ghi]terrylene-3,4:6,7:11,12-hexacarboxylic acid-3,4:6,7:11,12-trisimide 8 and N,N′,N″,N′″-Tetrakis-(1-nonyldecyl)dibenzo[ghi,tuv]terrylene-3,4:6,7:11,12:14,15-octacarboxylic acid-3,4:6,7:11,12:14,15-tetrakisimide 9

N,N′-Bis-(1-nonyldecyl)-3,4:11,12-terrylene tetracarboxylic bisimide 5 (35 mg, 33 micromol), maleic anhydride (80 mg, 0.82 mmol), chloranil (16 mg, 66 micromol) and nitrobenzene (10 mL) were stirred together for two hours at 210° C. bath temperature until a change of color from blue to purple was observed, followed by cooling down, pouring the reaction mass on 2 M HCl(50 mL), removal of nitrobenzene by water vapor distillation, degassing and drying at 110° C. The reaction mass was reacted without further purification with 1-nonyldecylamine (15 mg, 53 micromol) in imidazole (1.3 g) under argon at 140° C. for four hours. After cooling with a mixture of 2 M HCl and water free acetic acid (1:1, 20 mL) was added, the mixture was degassed and purified in a chromatography column (silicagel 60/CHCl₃). The first fraction was an orange coloured product mixture comprising compounds with structures 8 and 9 and some aliphatic side products (Rf =0.9, silica gel, chloroform). This first fraction was further fractionated by column chromatography (silicagel, isohexane). After removal of a first flow of an orange colored eluate, the eluating agent was changed to chloroform/isohexane 2:1. The orange colored product with formula 9 was first collected (2 mg, 4%), followed by the purple colored product with formula 8 (10 mg, 22%).

The products had the following characteristics:
Trisimide Product 8
R_{f}=0.5 (CHCl₃),
¹H NMR: (CDCl₃, 600 MHz, 25.0° C.): δ=10.51 (s, 1 H), 10.46 (d, ³J=16.5 Hz, 1 H), 9.41 (d, ³J=8.7 Hz, 1 H), 9.34 (d, ³J=8.6 Hz, 1 H), 9.15 (d, ³J=8.6 Hz, 1 H), 9.02 (d, ³J=8.1 Hz, 1 H), 8.75 (m, 3 H), 5.32 (m, 2 H), 5.23 (m, 1 H), 2.32 (m, 6 H), 1.91 (m, 6 H), 1.27 (m, 84 H), 0.87 ppm (m, 18 H),
UV/VIS: (CHCl₃): λₘₐₓ (Eᵣₑₗ): 584 (100), 539 (54), 503 (19), 415 (24), 393 nm (20),
Fluorescence (CHCl3): (CHCl₃): λₘₐₓ (Iᵣₑₗ): 595 (100), 647 nm (29),
Fluorescence quantum yield: (CHCl₃; E_{495 nm}=0.0211, λₑₓ=495 nm,
Reference: N,N′-Bis-(1-hexyheptyl)perylene-3,4:9,10-tetracarboxylic acid-3,4:9,10-bisimide with Φ=100%): Φ=100%,
MS: (DEP/EI): m/s (%): 1408 (60) [M⁺2x ¹³C], 1140 (40) [M⁺- C₁₉H₃₈],
873 (100) [M⁺-2x C₁₉H₃₈], 607 (52) [M⁺-3x C₁₉H₃₈], MS (FIA/ESI):
(C₉₅H₁₂₇N₃O₆) Calculated 1405.9693, Found. 1405.9667, Δ=−2.6 mmu.
Dibenzo Tetraimide Product 9
R_{f}=0.6 (CHCl₃),
UV/VIS: (CHCl₃): λₘₐₓ (Eᵣₑₗ): 519 (100), 483 (59), 452 (26), 399 nm (49),
Fluorescence: (CHCl₃): λₘₐₓ (Iᵣₑₗ): 530 (100), 574 nm (32),
Fluorescence quantum yield: (CHCl₃; E_{483 nm}=0.0306, λₑₓ=483 nm,
Reference: N,N′-Bis-(1-hexyheptyl)perylene-3,4:9,10-tetracarboxylic acid-3,4:9,10-bismide with Φ=100%): Φ=100%,
MS: (DEP/EI): m/s (%): 1766 (100) [M⁺2x ¹³C], 1500 (90) [M⁺- C₁₉H₃₈], 1233 (65) [M⁺-2x C₁₉H₃₈], 967 (18) [M⁺-3x C₁₉H₃₈], 701 (15) [M⁺-4x C₁₉H₃₈].
   The products 8 and 9 both had quantum yields of about 100%.

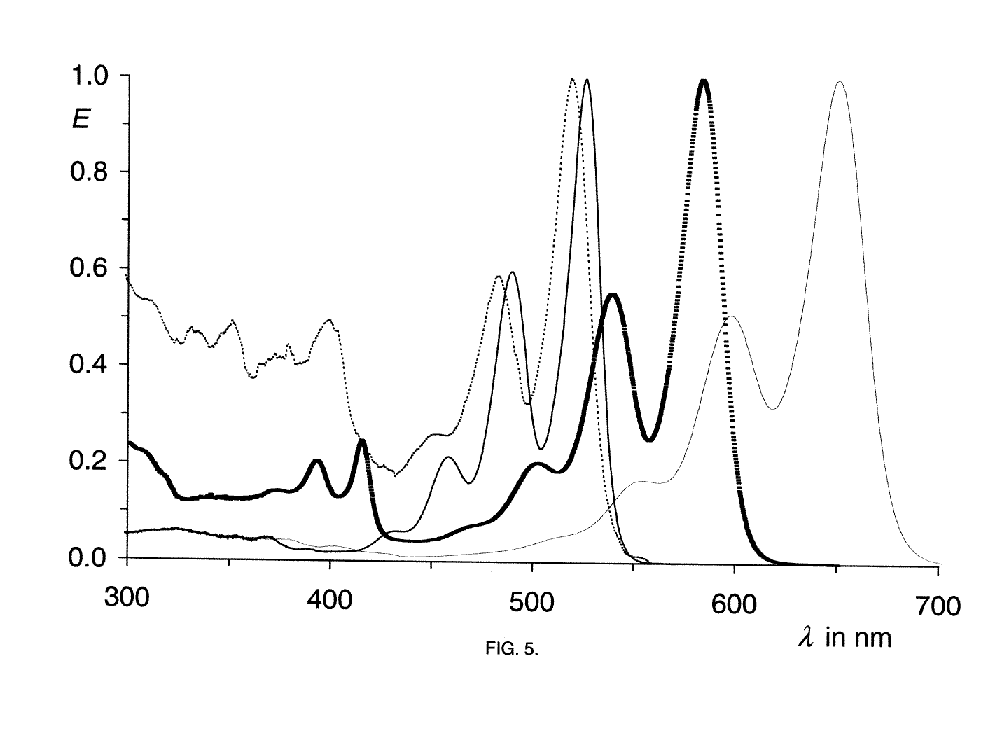

FIG. 5 shows the absorption spectra for the reference compound, N,N′-bis(1-hexylheptyl)-perylene-3,4:9,10-tetracarboxylic acid diimide, and compounds 5, 8, and 9. The reference compound is the heavy weighted line. Compound 5 is the full line. Compound 8 is the solid dash line. Compound 9 is the dofted line.

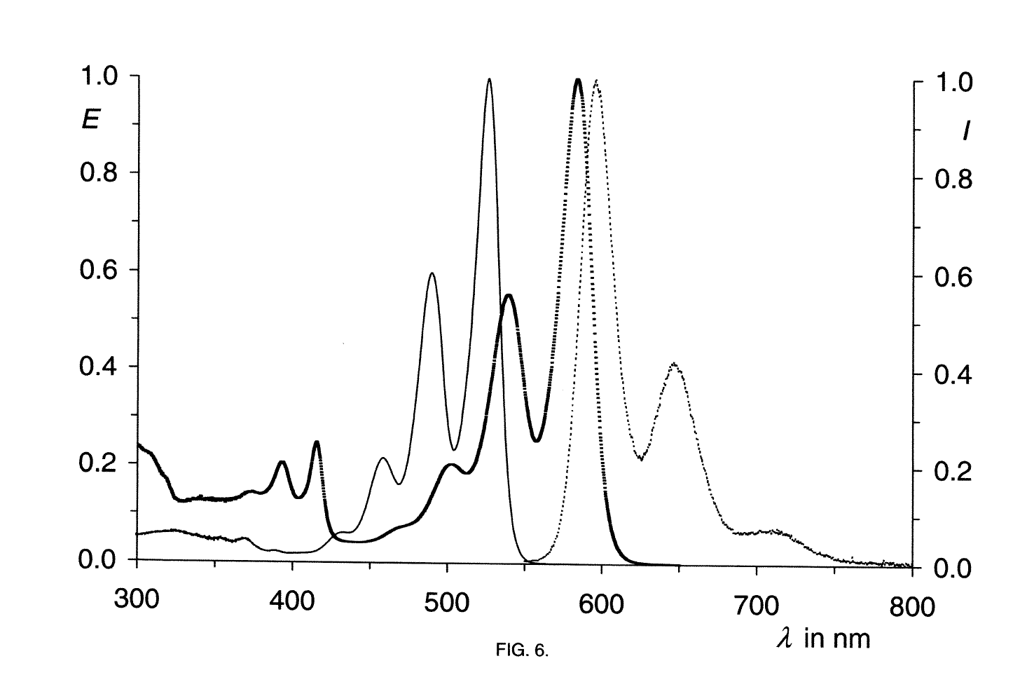

FIG. 6 shows the absorption spectrum for the reference compound and the absorption and emission spectra for compound 8. The reference compound is the full line. The absorption spectrum of compound 8 is the solid dash line. The emission spectrum of compound 8 is the dotted line.

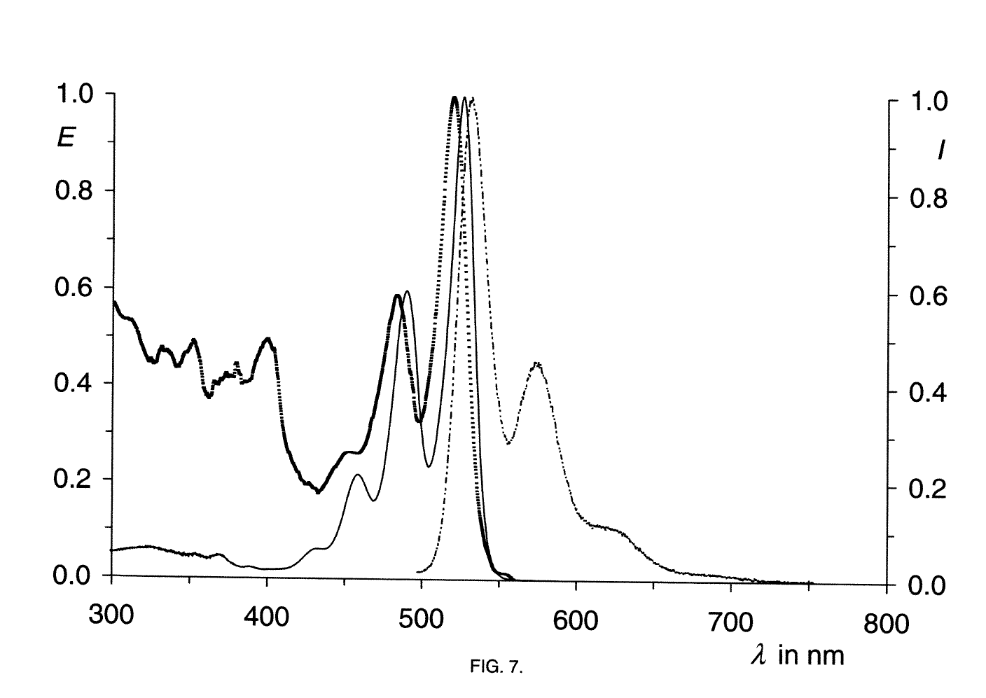

FIG. 7 shows the absorption spectrum for the reference compound and the absorption and emission spectra for compound 9. The reference compound is the full line. The absorption spectrum of compound 9 is the solid dash line. The emission spectrum of compound 9 is the dotted line.

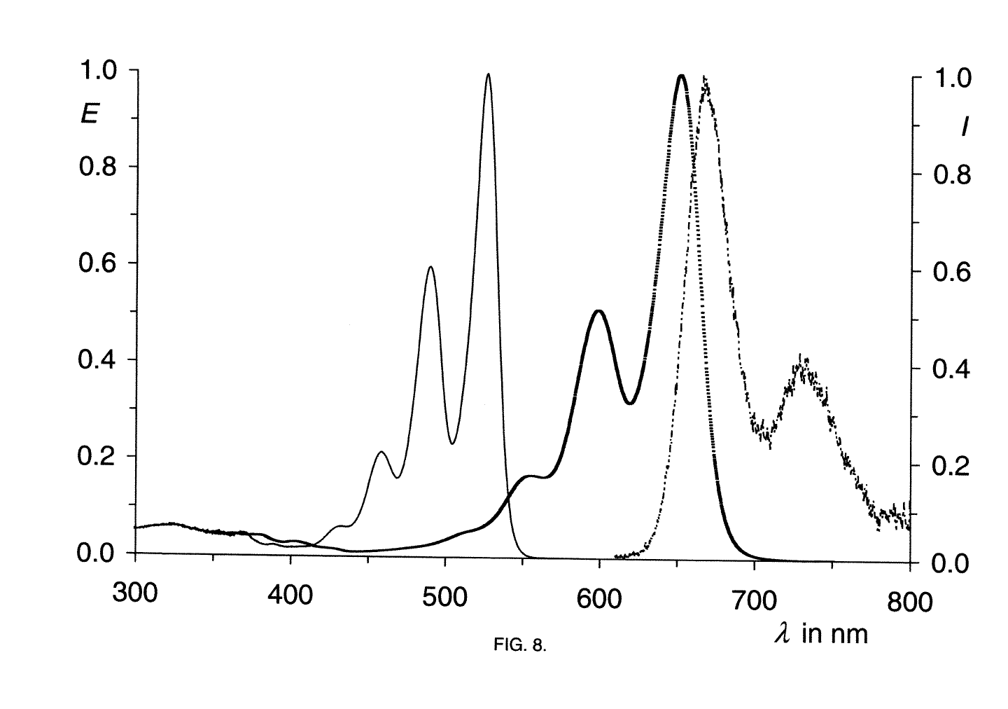

FIG. 8 shows the absorption spectrum for the reference compound and the absorption and emission spectra for compound 5. The reference compound is the full line. The absorption spectrum of compound 5 is the solid dash line. The emission spectrum of compound 5 is the dofted line.

The benzoterrylenes of the present disclosure have been described with reference to exemplary embodiments. Obviously, modifications and alterations will occur to others upon reading and understanding the preceding detailed description. It is intended that the exemplary embodiments be construed as including all such modifications and alterations insofar as they come within the scope of the appended claims or the equivalents thereof.

1. A benzoterrylene of Formula (I): 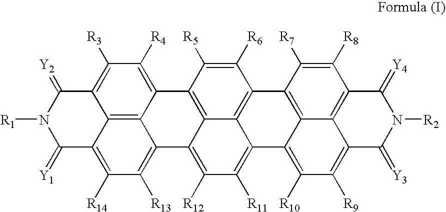
wherein at least one of the pairs R₄-R₅, R₆-R₇, R₁₀-R₁₁, and R₁₂-R₁₃ is a ring structure selected from the group consisting of Formulas A, B, and C: 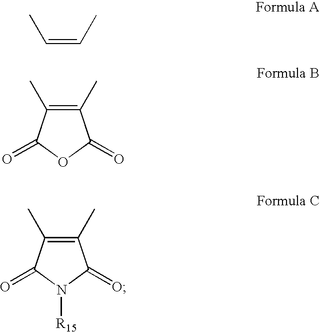
wherein Y₁ through Y₄ are each independently selected from O and NR₁₆, and R₁ or R₂ may independently combine with R₁₆ to form a ring structure selected from the group consisting of Formulas D and E: 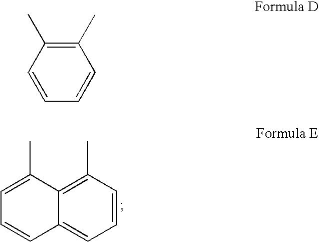
wherein R₁, R₂, R₁₅, and R₁₆ are each independently selected from hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono; and
wherein R₃ through R₁₄ are each independently selected from halogen, cyano, hydroxyl, hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono.

2. The benzoterrylene of claim 1, wherein the benzoterrylene is of Formula (II):

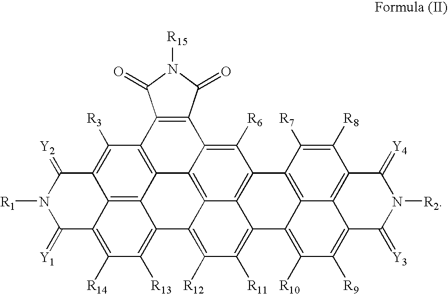

3. The benzoterrylene of claim 1, wherein the benzoterrylene is of Formula (IlI):

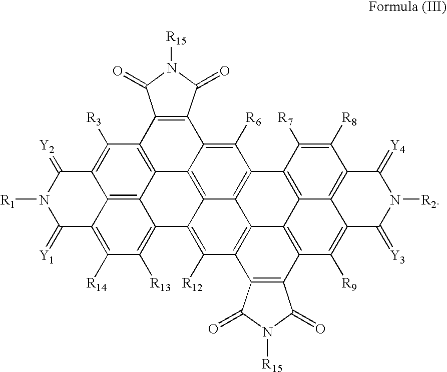

4. The benzoterrylene of claim 1, wherein the benzoterrylene is of Formula (IV):

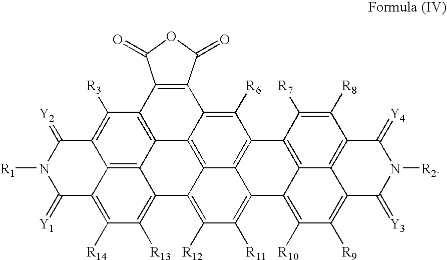

5. The benzoterrylene of claim 1, wherein the benzoterrylene is of Formula (V):

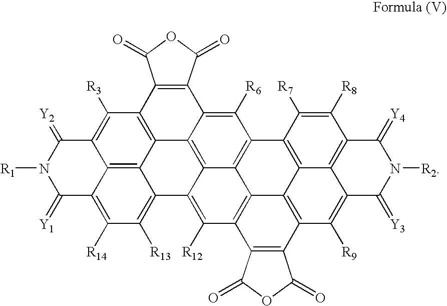

6. The benzoterrylene of claim 1, wherein the benzoterrylene is of Formula (VI): 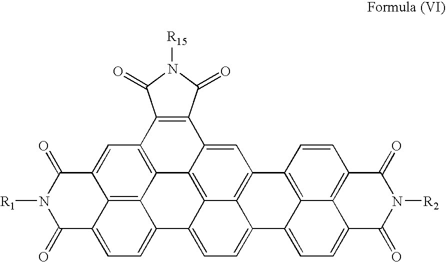
wherein R₁, R₂, and R₁₅ are independently selected from secondary alkyl having from about 10 to about 25 carbon atoms.

7. The benzoterrylene of claim 1, wherein the benzoterrylene is of Formula (VII): 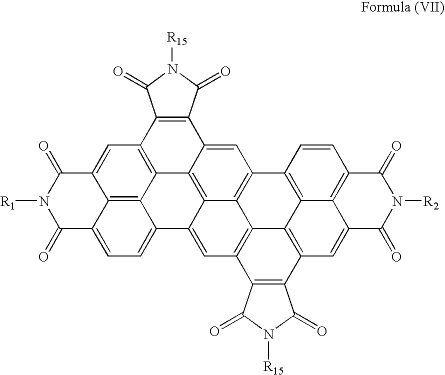
wherein R₁, R₂, and each R₁₅ are independently selected from secondary alkyl having from about 10 to about 25 carbon atoms.

8. The benzoterrylene of claim 1, wherein the benzoterrylene is of Formula (VIII): 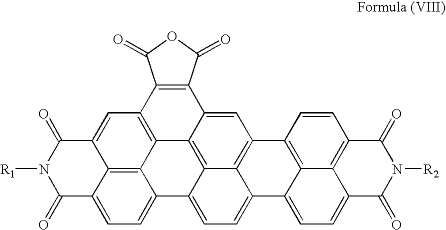
wherein R₁ and R₂ are independently selected from secondary alkyl having from about 10 to about 25 carbon atoms.

9. The benzoterrylene of claim 1, wherein the benzoterrylene is of Formula (IX): 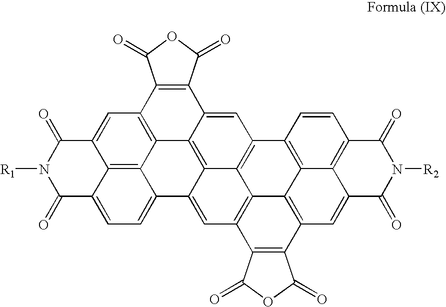
wherein R₁ and R₂ are independently selected from secondary alkyl having from about 10 to about 25 carbon atoms.

10. The benzoterrylene of claim 1, having a fluorescent quantum yield of at least 70%.

11. A composition comprising a polymeric resin and the benzoterrylene of claim 1.

12. A dye or pigment, comprising the benzoterrylene of claim 1.

13. A method of coloring a polymeric resin, comprising the step of incorporating the benzoterrylene of claim 1 into the polymeric resin.

14. An article molded from a composition, the composition incorporating the benzoterrylene of claim 1.

15. A luminescent solar collector, comprising:
a sheet which comprises a polymer and the benzoterrylene of claim 1; and
a light energy converter which is operatively connected to the sheet.

16. A method of preparing a benzoterrylene of Formula (I): 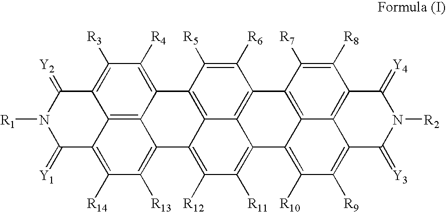
wherein at least one of the pairs R₄-R₅, R₆-R₇, R₁₀-R₁₁, and R₁₂-R₁₃ is a ring structure selected from the group consisting of Formulas A, B, and C: 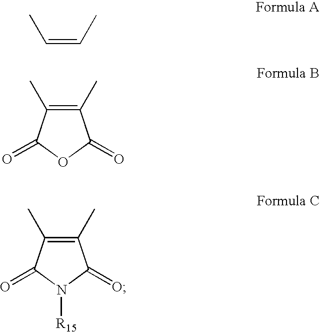
wherein Y₁ through Y₄ are each independently selected from O and NR₁₆, and R₁ or R₂ may independently combine with R₁₆ to form a ring structure selected from the group consisting of Formulas D and E: 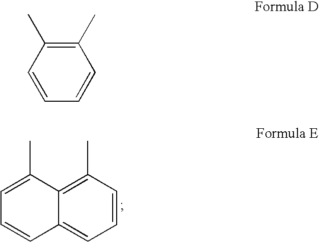
wherein R₁, R₂, R₁₅, and R₁₆ are each independently selected from hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono; and
wherein R₃ through R₁₄ are each independently selected from halogen, cyano, hydroxyl, hydrogen, alkyl, cycloalkyl, aryl, aryloxy, thiophenyl, carbonylalkyl, carbonylphenyl, alkyl carboxylic acid, or carboxylalkyl, each of which may be further substituted with alkyl, cycloalkyl, aryl, aryloxy, thiophenyl, carbonylalkyl, carbonylphenyl, alkyl carboxylic acid, carboxylalkyl, halogen, cyano, oxo, nitrogen, hydroxyl, epoxy, amino, carboxyl or thiono,
the method comprising:
reacting a naphthalene-1,8-dicarboximide of the general formula 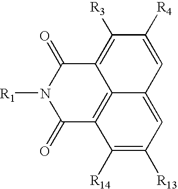
with a perylene-3,4-dicarboximide of the general formula 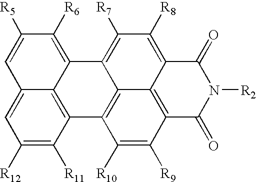
to form a terrylene tetracarboxylic bisimide; and
reacting the terrylene tetracarboxylic bisimide with a dienophile to obtain the benzoterrylene.

17. The method of claim 16, wherein the dienophile is an unsaturated dicarboxylic acid or a dicarboxylic acid anhydride.

18. The method of claim 17, further comprising the step of decarboxylating the benzoterrylene to obtain a benzoterrylene of Formula A.

19. The method of claim 16, wherein the dienophile is maleic acid.

20. The method of claim 16, further comprising reacting the benzoterrylene with an amine to obtain a benzoterrylene hexacarboxylic trisimide.

21. The method of claim 16, wherein the naphthalene-1,8-dicarboximide and perylene-3,4-dicarboximide are reacted at atmospheric pressure at a temperature of from about 130° C. to about 160° C. for at least three hours to form the terrylene tetracarboxylic bisimide; and
wherein the terrylene tetracarboxylic bisimide and dienophile are reacted in a nitrobenzene solution at atmospheric pressure at a temperature above 200° C. for more than two hours.
